(19) United States
(12) Patent Application Publication
(10) Pub. No.: US 2026/0217631 A1
(43) Pub. Date: Jul. 30, 2026

(54) ENERGY-EFFICIENT PROCESS FOR SEPARATING BUTENES FROM C4-HYDROCARBON STREAMS

(71) Applicant: EVONIK OXENO GMBH & CO. KG, Marl (DE)

(72) Inventors: Niklas PAUL, Marl (DE); Armin Matthias RIX, Marl (DE); Moritz SCHRÖDER, Münster (DE); Tanita Valèrie SIX, Dortmund (DE); Andreas OLDENKOTT, Haltern am See (DE); Benjamin WOLDT, Südlohn (DE); Thomas BECK, Recklinghausen (DE); Dietmar HOHMANN, Marl (DE); Stefan VOLLE, Dülmen (DE); Roland BRANGES, Marl (DE)

(73) Assignee: EVONIK OXENO GMBH & CO. KG, Marl (DE)

(21) Appl. No.: 19/137,368

(22) PCT Filed: Dec. 7, 2023
(86) PCT No.: PCT/EP2023/084759

§ 371 Date: Jun. 10, 2025

(30) Foreign Application Priority Data
Dec. 12, 2022 (EP) 22212705.2

(51) Int. Cl. C07C 7/08; B01D 3/14; B01D 3/40

(52) U.S. Cl. CPC C07C 7/08; B01D 3/143; B01D 3/40

(57) ABSTRACT

The invention relates to a process for separating butenes from C4-hydrocarbon streams containing butanes as well as butenes by means of extractive distillation with a suitable solvent. The process according to the invention is characterized in that in the inlet region of at least one packed bed of the absorber there are packing elements of lower specific surface area than in the rest of the packed bed.

The present invention relates to a process for separation of butenes from C4-hydrocarbon streams containing not only the butenes but also butanes by extractive distillation with a suitable solvent. The process according to the invention has the feature that the inlet region of at least one random-packing bed of the absorber comprises random packings of lower specific surface area than the remainder of the random-packing bed.

The separation of butane-butene mixtures by extractive distillation is known per se. This employs an aprotic solvent (for example N-methyl-2-pyrrolidone (NMP) or acetonitrile (ACN)) to increase the relative volatility of the alkanes compared to the alkenes. In one extractive distillation column, the absorber, the butenes are preferably dissolved in the solvent and the butanes are separated as tops product. The laden solvent is subsequently freed of the butenes in a stripping column, the desorber, at elevated temperature and/or reduced pressure, and said butenes are obtained in enriched form as tops product. The solvent freed of butenes is then recycled to the extractive distillation. A typical process for separating butane-butene mixtures by extractive distillation is described in WO 2022/161869 A1 for example.

In all process variants the butenes are washed out of the butane-butene mixtures, i.e. the employed C4-hydrocarbon streams, with an initially liquid solvent in the absorber column. If the process is implemented as described in the prior art it must be noted that in the inlet regions of the random-packing beds of the absorber temperature and/or concentration gradients can lead to the occurrence of a miscibility gap or to condensation of fine droplets. This can lead to fluid accumulation in the inlet regions of the random-packing beds of the absorber, thus resulting in premature blockage of the absorber column. This reduces the separation efficiency in these regions.

The processes described in the prior art can thus have the problem that the separation efficiency in the inlet regions is reduced. Overall, this results in a reduction in plant capacity and in the need to use greater amounts of solvents.

The problem addressed by the present invention is accordingly that of providing a process where an improved separation efficiency of the absorber may be achieved in the simplest possible way. A further problem addressed was that of reducing the amount of solvent to be employed.

This problem is solved by the embodiment of the process proposed in claim 1. Preferred embodiments are specified in the dependent claims. The process according to the invention is a process for separation of butenes from a C4-hydrocarbon stream which contains at least butenes and butanes by extractive distillation with a solvent, wherein the process comprises the steps of:
a. supplying the gaseous C4-hydrocarbon stream and the liquid solvent, preferably NMP, to an absorber which comprises at least three random-packing beds arranged one below the other and in which the C4-hydrocarbon stream and the solvent are contacted with one another to transfer predominantly butenes from the C4-hydrocarbon stream into the solvent to form a laden solvent, wherein the thus-laden solvent is collected in a liquid collector of the absorber and passed through an absorber evaporator and then passed into the bottom of the absorber below the liquid collector to outgas predominantly butanes from the laden solvent and wherein the laden solvent is subsequently passed to a desorber as bottoms stream;
b. supplying the laden solvent to the desorber, the bottom of which is at an elevated temperature and preferably a lower pressure relative to the bottom of the absorber and in which the butenes are separated from the solvent to obtain at the top of the desorber a stream enriched in butenes, wherein a solvent, at least partially freed of butenes, is collected in a liquid collector of the desorber and passed through a desorber evaporator and then passed into the bottom of the desorber below the liquid collector to outgas any butenes still remaining in the solvent and wherein the solvent is subsequently recycled to the absorber as bottoms stream;
   characterized in that at least one of the random-packing beds of the absorber contains different random packings, wherein the random packings in the upper region of the random-packing bed have a specific surface area y and the random packings in the lower region of the random-packing bed have a specific surface area x, wherein the specific surface area x is greater than the specific surface area y.

The solution according to the invention is accordingly that of employing two random packings having different specific surface areas in at least one random-packing bed of the absorber. The random-packing beds are typically filled with a dumped bed of the random packings. The random-packing bed is filled initially with the random packings having the greater specific surface area x and subsequently with the random packings having the lower specific surface area. The random packings of specific surface area y which form the upper region are thus disposed on the random packings of specific surface area x which form the lower region. The difference in the specific surface area of random packings for extraction distillation columns according to the invention is based for example on the size of the random packings. Based on a volume into which the random packings are filled, large random packings are known to have a smaller specific surface area than small random packings.

Having regard to the specific surface areas x and y of the random packings a limitation to exact numerical values/threshold values is impossible. The surface areas to be employed depend on the spatial configuration of the extraction distillation system or on the composition of the employed hydrocarbon stream. However, the specific surface area x of the random packings in the lower region of the at least one random-packing bed is preferably at least 10% higher than the specific surface area y of the random packings in the upper region of the at least one random-packing bed.

The inlet region of the random-packing beds, in particular of the uppermost beds, features a pronounced temperature and concentration profile. Here, cold unladen solvent is added to a hot gas stream, thus resulting in condensation of the gas phase. There is also a high risk of forming a second liquid phase at this point. The formation of fine droplets through concentration or temperature profiles ensures that droplets are kept in suspension by the gas stream and do not immediately flow away. Consequently the liquid burden in the inlet region of the random-packing beds is higher than in the remainder of the random-packing bed and accumulation of the liquid, so-called flooding, occurs. Flooding in the inlet region leads to a hydraulic bottleneck and also to a loss of separation efficiency. It has been found that the use of two different random packings in one random-packing bed of the absorber in the upper portion of the absorber leads to capacity and separation efficiency gains.

The exact spatial configuration of the random-packing beds comprising the different random packings in the configuration according to the invention depends on the configuration of the absorber. Thus, apart from the fact that the random packings of lower specific surface area y are arranged above the random packings of greater specific surface area x, the exact implementation may be varied and may be adapted to the conditions prevailing in each case. However, it is in principle the case that preferably a larger portion of the volume of the random-packing bed should be filled with the random packings of specific surface area x, i.e. the random packings having the larger surface area. In this context it is preferable when not more than 20% of the volume of the random-packing bed is filled with the random packings of specific surface area y. The remaining 80% of the volume is then allocated to the random packings of specific surface area x. When installing the random packings it may be preferable to observe the fill height of the random packings in the random-packing bed. In this context it is preferable when the random packings of specific surface area y account for 20% of the fill height of the random-packing bed. The remaining 80% of the fill height is then allocated to the random packings of specific surface area x.

The absorber of the process according to the invention comprises at least three random-packing beds. According to the invention it is preferable when the at least one random-packing bed having the different random packings is the uppermost random-packing bed of the at least three random-packing beds of the absorber. However, it is also possible for two or three random-packing beds of the at least three random-packing beds of the absorber to contain the different random packings in the configuration according to the invention. Particular preference is given to the embodiment where all random-packing beds in the absorber are provided with different random packings in the configuration according to the invention.

The absorber may also contain more than three random-packing beds. If the absorber comprises more than three random-packing beds it is preferable when the at least one random-packing bed having the different random packings is the uppermost random-packing bed of the at least three random-packing beds of the absorber. However, it is also possible for two or three random-packing beds of the at least three random-packing beds of the absorber to contain the different random packings in the configuration according to the invention. In a preferred embodiment of the present invention the two or three random-packing beds comprising different random packings in the configuration according to the invention are the uppermost two or the uppermost three random-packing beds of the absorber. Particular preference is given to the embodiment where all random-packing beds in the absorber are provided with different random packings in the configuration according to the invention.

The present process relates to the separation of butenes from butene-containing C4-hydrocarbon streams. These streams typically also contain alkanes (n-butane, isobutane) in addition to the butenes. In the context of the present invention-unless something else is being described-the term butanes is to be understood as meaning both n-butane and isobutane. The process according to the invention may therefore employ all C4-hydrocarbon streams which contain at least butenes and butanes provided that the amounts in which the butenes and/or butanes are present allow economic performance of the process. In a preferred embodiment of the present invention the employed C4-hydrocarbon stream consists essentially, i.e. to an extent of more than 98% by weight, preferably to an extent of more than 99% by weight, of butanes and butenes. The corresponding streams may also contain impurities or other hydrocarbons, such as 1,3-butadiene or C5-hydrocarbons, in small amounts.

The extraction process according to the invention employs a liquid solvent which dissolves primarily the butenes of the employed gaseous C4-hydrocarbon stream. Suitable solvents are aprotic solvents, for example N-methyl-2-pyrrolidone (NMP). The process according to the invention is preferably performed with NMP as the solvent. In a further preferred embodiment of the present invention the solvent contains water, in particular in the range from 1% to 10% by weight, preferably from 4% to 9% by weight, in each case based on the total amount of solvent.

In the context of the present invention the absorber employed is a random-packing column comprising at least three random-packing beds arranged one below the other. Such columns are known in principle to those skilled in the art. Preferably arranged above the first random-packing column is a backwashing zone comprising a plurality of theoretical trays to hold back the solvent entrained in the gas phase. Above the backwashing zone is the top of the absorber where a stream enriched in butanes relative to the employed C4-hydrocarbon stream is obtained. The liquid collector according to the invention would be arranged below the last random-packing bed, the bottom of the absorber being arranged below said collector. The precise construction of the absorber depends on various parameters and is variable in certain aspects.

The liquid solvent is preferably supplied to the absorber spatially above the inlet for the C4-hydrocarbon stream. In a preferred embodiment the solvent is supplied to the absorber above the first random-packing bed and the C4-hydrocarbon stream is supplied to the absorber in one or more random-packing beds below the first random-packing bed. In the absorber the liquid solvent trickles downwards and is brought into contact with the (ascending) vaporous C4-hydrocarbon stream to transfer a portion of the C4-hydrocarbon stream containing predominantly butenes to the solvent to form a laden solvent. In step a the C4-hydrocarbon stream and the solvent are thus brought into contact with one another in particular in countercurrent. In a preferred embodiment of the present invention the portion of the C4-hydrocarbon stream transferred to the solvent comprises at least 70% by weight, particularly preferably at least 80% by weight, of butenes, based on the composition of the portion of the C4-hydrocarbon stream transferred to the solvent. This has the result that in particular at least 80%, particularly preferably at least 90%, of the butenes present in the employed C4-hydrocarbon stream are transferred to the solvent.

The laden solvent flows downwards in the absorber and is collected in a suitable liquid collector, in particular a chimney tray. The temperature of the laden solvent accumulating in the liquid collector is preferably between 40° C. and 90° C., particularly preferably between 45° C. and 65° C. The laden solvent is withdrawn from the liquid collector, passed through an absorber evaporator and then passed into the bottom of the absorber below the liquid collector to outgas predominantly butanes from the laden solvent. The absorber evaporator is preferably a once-through evaporator where the laden solvent is passed through the evaporator only once. This makes it possible to achieve the lowest possible temperatures, thus making it possible to prevent fouling. The driving temperature difference is additionally increased, thus allowing even more efficient energy utilization of the NMP stream. The absorber evaporator may also be configured as a multi-stage apparatus, i.e. a plurality of heat exchangers/a plurality of evaporators belonging to the absorber evaporator may be present. The solvent laden predominantly with butenes then remains in the bottom and is withdrawn therefrom and passed to the desorber as bottoms stream. The temperature in the bottoms stream of the absorber which is passed to the desorber is preferably between 70° C. and 130° C., particularly preferably between 85° C. and 120° C.

A stream enriched in butanes compared to the employed C4-hydrocarbon stream is then especially obtained at the top of the absorber. The overhead pressure in the absorber may be between 3 and 7 bar absolute, preferably between 4 and 6.5 bar absolute. The stream enriched in butanes may additionally contain water originating from the solvent. This water may be separated in a subsequent step. The stream enriched in butanes is withdrawn at the top of the absorber and preferably subjected to a single-or multi-stage condensation to condense out a water-containing stream and a butane-containing product stream. These two streams may be separated from one another in a suitable apparatus, for example a spider. The water-containing stream separated from the butane-containing product stream may be passed to the absorber or to the desorber and/or partially discharged from the process depending on its composition.

Depending on the requirements for the obtained butane-containing product stream it may be necessary for the butane-containing product stream to be subjected after the condensation to a drying, preferably in a drying column, to separate the water still present. The butane-containing product stream preferably contains a maximum amount of water of 50 ppmw, preferably of 25 ppmw, after the drying. The water obtained during the drying may be recycled to the condensation in the absorber.

The solvent withdrawn at the bottom of the absorber and laden predominantly with butenes is supplied to the desorber. To this end the laden solvent may be passed to the desorber using a pump for example. Relative to the bottom of the absorber the bottom of the desorber is at an elevated temperature and preferably a lower pressure. The temperature in the bottom of the desorber is preferably between 120° C. and 200° C., more preferably between 130° C. and 195° C. The head pressure in the desorber may be between 1 and 6 bar absolute, preferably between 2 and 5 bar absolute. The elevated temperature and the preferably lower pressure relative to the absorber has the result that the butenes and any butanes still present are at least partially removed from the solvent. In a preferred embodiment the solvent at least partially freed of butenes contains up to 5000 ppmw of butenes, particularly preferably 100 to 900 ppmw of butenes. The solvent at least partially freed of butenes flows downwards in the desorber and is collected in a liquid collector of the desorber. From there the solvent at least partially freed of butenes is passed through a desorber evaporator and then passed into the bottom of the desorber below the liquid collector, in particular a chimney tray, to outgas any butenes still remaining in the solvent. The desorber evaporator is preferably a once-through evaporator where the solvent at least partially freed of butenes is passed through the evaporator only once. This makes it possible to achieve the lowest possible temperatures, thus making it possible to prevent fouling. The desorber evaporator may also be configured as a multi-stage apparatus, i.e. a plurality of heat exchangers belonging to the desorber evaporator may be present. The solvent freed of butenes then remains in the bottom and is withdrawn therefrom, passed to the absorber as bottoms stream and reused there as solvent for the absorption of butenes.

Before being passed to the absorber the solvent freed of butenes may be partially or completely subjected to a regeneration to remove impurities, for example the abovementioned by-products present in the employed C4-hydrocarbon stream and/or formed from the butenes at the temperatures in the desorber such as oligomeric or polymeric compounds, from the solvent, preferably the NMP. The regeneration is preferably performed such that the solvent freed of butenes is passed into a container and evaporated at a pressure of less than 500 mbar absolute, more preferably of less than 200 mbar absolute and a temperature between 100° C. and 150° C. The container may have a column connected to it. Heavy boilers in particular are separated by the regeneration. If only a portion of the solvent freed of butenes is subjected to a regeneration the regenerated portion of the solvent is subsequently combined with the unregenerated solvent and recycled to the absorber.

The process according to the invention may preferably further feature heat integration with which the heat of the solvent is utilized for heating and/or at least partially evaporating different streams. The heat of the solvent, preferably of the NMP, withdrawn as a bottoms stream of the desorber is at least partially used for heat integration by employing the heat of the solvent, preferably of the NMP, in at least one respective heat exchanger for preheating the laden solvent, preferably NMP, passed to the desorber, for evaporation in the absorber evaporator and for evaporation of the liquid C4-hydrocarbon stream in a feed evaporator.

One advantage is the simple construction of the heat integration which nevertheless allows efficient energy recovery. The simplest embodiment of the present invention further does not mandate additional side evaporators which entail additional plant engineering complexity and thus higher costs.

The heat integration removes heat from the solvent. The reason for this is not only that other streams or columns are to be heated but rather primarily the cooling of the solvent for the absorption. The absorption of the butenes (here: step a) is usually carried out at a lower temperature than the desorption (here: step b). If in the course of the heat integration sufficient heat is withdrawn from the solvent, i.e. it has a suitable temperature, the solvent may be passed directly into the absorber. However, it is also conceivable that despite the heat integration present the solvent does not yet have the correct temperature. In such a case the solvent may be passed through a residual cooler to be cooled to a suitable temperature after the heat integration and before entry into the absorber.

Heat is a process parameter. The heat supplied or removed corresponds to the change in internal energy minus the work done. The terms heat, heat transport and heat integration used in the present invention are always based on this definition.

In a preferred embodiment of the present invention the preheating of the laden solvent passed to the desorber is performed in two stages, wherein a first heat transfer to the laden solvent passed to the desorber is effected in a heat exchanger, for example a tube bundle heat exchanger, and a second heat transfer to the laden solvent passed to the desorber is effected in a kettle evaporator. Such an embodiment has the advantage that in the abovementioned preferred embodiment the heat transferred to the laden solvent in both stages, i.e. in the heat exchanger and in the kettle evaporator, originates from the solvent withdrawn as the bottoms stream of the desorber as heat transfer medium. The use of a kettle evaporator also has the advantage that it would allow there to be a lower supply pressure in the conduit to the desorber. A high supply pressure is normally necessary to prevent evaporation in the pipe conduit that could lead to problems including bursting of the pipe conduit. A further advantage is that the thermal load is limited, thus ensuring that the temperature difference is/remains sufficiently large for heat transfer.

A stream enriched in butenes compared to the employed C4-hydrocarbon stream is then especially obtained at the top of the desorber. This stream enriched in butenes may additionally contain water originating from the solvent. This water may be separated in a subsequent step. The stream enriched in butenes is withdrawn at the top of the desorber and subjected to a single-or multi-stage condensation to condense out a water-containing stream which may contain not only water but also residues of organics as well as a butene-containing product stream. These two streams may be separated from one another in a suitable apparatus, for example a spider. The water-containing stream separated from the butene-containing product stream may then be recycled to the desorber. Discharging the entirety or portions of the water-containing stream to remove the organics is also possible.

The butene-containing product stream obtained from the condensation preferably contains less than 20% by weight, more preferably less than 16% by weight, of butanes based on the total composition of the butene-containing product stream. By contrast, the butene-containing product stream obtained from the condensation preferably has a butene content of at least 70% by weight, more preferably of at least 75% by weight, particularly preferably of at least 86% by weight, based on the total composition of the butene-containing product stream.

According to the invention the solvent, preferably NMP, at least partially freed of butenes is collected in a liquid collector of the desorber and passed through a desorber evaporator to allow outgassing of any butenes still remaining in the solvent. The heat for evaporation in the desorber evaporator may be introduced in a heat exchanger by heat transfer from a suitable heat transfer medium. The heat transfer medium may be in particular heating steam employed in the form of medium pressure or high pressure steam. A preferred heating steam is a medium pressure steam having a temperature of 150° C. to 270° C., preferably of 160° C. to 250° C. The medium pressure steam preferably has a pressure of 15 to 30 bar absolute, particularly preferably of 17 to 25 bar absolute. Also employable as heating steam is a steam having a pressure of >30 bar absolute. Such a heating steam may also be referred to as high pressure steam.

The heating steam used for evaporation may undergo at least partial condensation in the heat exchanger, thus generating a hot condensate at a pressure of 10 to 20 bar absolute, preferably 12 to 17 bar absolute, and a temperature of 150° C. to 210° C., preferably 160° C. to 200° C. Preferably arranged downstream of the heat exchanger is a condensate container in which the hot condensate may be separated from the steam. The pressure in the condensate container is preferably lower than in the heat exchanger on the heating steam side. The lower pressure may result in a portion of the hot condensate evaporating, as a result of which the combined steam, i.e. the uncondensed proportion of the heating steam and the hot condensate evaporated in the condensate container by decompression, is obtained as low pressure steam in the condensate container. In the present case low pressure steam preferably has a pressure of more than 0 bar and less than 10 bar absolute. The temperature of the low pressure steam is preferably 100° C. to 180° C.

The low pressure steam obtained there still contains energy but this is not utilized in any known process. However, this is not advantageous from an energetic or economic standpoint. This energy can, however, be utilized in a preferred embodiment of the present invention. To this end, the heating steam used for evaporation in the desorber evaporator may be provided using a, preferably controllable, steam ejector (thermocompressor). The thermocompressor is then supplied both with the employed heating steam, originating from an appropriate steam network for example, here especially the preferably employed medium pressure steam, and also with the low pressure steam from the condensate container, thus forming a mixed steam which is accordingly the heat transfer medium for the desorber evaporator. In this embodiment the mixed steam is accordingly the heating steam. Such a steam ejector is configured such that it is operated with a motive steam and by means of a negative pressure (back pressure in the steam ejector) can aspirate suction steam from a container to then form the mixed steam employed as the heat transfer medium. The motive steam is in the present case the heating steam or the medium pressure steam, with which the low pressure steam is aspirated from the condensate container as suction steam and mixed with the motive steam.

The advantage of such an embodiment is obvious. The energy of the low pressure steam obtained in the condensate container may be utilized, thus saving energy and costs. Such a procedure can also be advantageous for another reason. The employed steam ejector may be controllable such that the amounts of medium pressure/high pressure and low pressure steam can be adjusted, for example according to particular process parameters. The amount of suction steam is adjusted via the amount of motive steam. The amounts of low pressure and medium pressure steam may be adjusted for example according to the temperature in the desorber.

In a further preferred embodiment the desorber comprises a side evaporator. In such a case the heat transfer medium used for the side evaporator may be the mixed steam from the steam ejector while the desorber evaporator employs medium pressure steam as heating steam. The hot condensate from the desorber evaporator and the side evaporator are then passed to a condensate container as described hereinabove. The low pressure steam obtained there is then used in the steam ejector, whose mixed steam is employed in the side evaporator. The advantage of this variant is that the hot condensate obtained can be further decompressed to provide a greater amount of low pressure steam.

The presently described process may be employed in integrated chemical systems which comprise in particular an oligomerization and optionally a hydroformylation. It is possible for the separation of butenes by the process according to the invention to be employed at various points in the integrated system. It is also possible for the separation of butenes according to the invention to be present at various points within an integrated chemical system. For example the process described here may be employed at the beginning of such an integrated system. The employed C4-hydrocarbon stream may then be in particular a crack C4, a raffinate 1, a raffinate 2 or a mixture thereof. If crack C4 and/or raffinate 2 are employed the separation process according to the invention may have arranged upstream of it a crack C4 hydrogenation in which butadiene is selectively hydrogenated or a butadiene separation in which butadiene is extractively removed with a solvent such as NMP or nitriles to reduce the content of butadiene. It is also possible to arrange a hydroisomerization downstream of an extractive butadiene separation and upstream of the separation according to the invention to facilitate the separation task in the process according to the invention since this converts 1-butene into 2-butene which is generally more readily absorbed by the solvent.

If the separation process is employed at the beginning of the integrated system the obtained product stream may be supplied to an MTBE synthesis which may then preferably be followed successively by a 1-butene separation, an oligomerization and one or more hydroformylations of the purified oligomers. A hydroformylation may be carried out not only with the product stream from the oligomerization, thus making it possible to produce for example INA (isononanol) from di-n-butenes after subsequent hydrogenation or ITDA (isotridecanal) from tributenes, but also with the unconverted butenes of the oligomerization, thus making it possible to produce 2-PH (2-propylheptanol) after subsequent aldol condensation followed by hydrogenation. The unconverted butenes from the oligomerization could optionally also be used to operate a further oligomerization instead of a hydroformylation. The conditions of the individual process steps are familiar to those skilled in the art. The individual process steps may comprise further steps, such as for example the separation of the products or the workup of the resulting streams, but these are not explicitly mentioned here. However, the separation process according to the invention may also be introduced at any other point of such an integrated system.

In one embodiment of the present invention the C4 hydrocarbon stream employed in the separation process according to the invention is withdrawn from an MTBE synthesis after separation of MTBE and the butene-containing product stream is subsequently supplied to a 1-butene separation, after which an oligomerization and one or more hydroformylations for subsequent production of 2-PH, ITDA and/or INA are successively carried out. The individual process steps may comprise further steps, such as for example the separation of the products or the workup of the resulting streams, but these are not explicitly mentioned here.

In a further embodiment of the present invention the C4-hydrocarbon stream employed in the separation process according to the invention is withdrawn from a 1-butene separation and the butene-containing product stream is subsequently supplied to an oligomerization, after which one or more hydroformylations for subsequent production of 2-PH, ITDA and/or INA are carried out. The individual process steps may comprise further steps, such as for example the separation of the products or the workup of the resulting streams, but these are not explicitly mentioned here.

In a further embodiment of the present invention the C4-hydrocarbon stream employed in the separation process according to the invention is withdrawn from an oligomerization and the butene-containing product stream is subsequently supplied to a hydroformylation for subsequent production of 2-PH. The individual process steps may comprise further steps, such as for example the separation of the products or the workup of the resulting streams, but these are not explicitly mentioned here.

In a further embodiment of the present invention the separation process according to the invention is employed at the end of the integrated system. In that case the employed C4-hydrocarbon stream is withdrawn from a 2-PH production downstream of the hydroformylation. The butene-containing product stream then obtained from the separation process according to the invention may in this case be recycled and employed at a suitable point in the integrated system, for example for 1-butene separation, for oligomerization or one or more hydroformylations. This makes it possible to enhance the efficiency of the overall integrated system since even after passing through the last process step in the integrated system up to 20% by weight of butenes may still be present.

Independently of the point in the integrated system where the separation process according to the invention is arranged the butane-containing product stream may be supplied for example to an adiabatic oligomerization, a hydrogenation of the butenes still present or an n/iso splitting of the butanes where n-butane and isobutane are separated from one another. The n/iso splitting may also be carried out after an adiabatic oligomerization. Another possibility would be inclusion of the butane-containing product stream upstream of the oligomerization in an above-described integrated system composed of MTBE synthesis, 1-butene separation, oligomerization and a hydroformylation.

In a particularly preferred embodiment of the present invention the energy required for n/iso splitting may be effected at least partially by heat integration with the first stage of a two-stage condensation at the top of the desorber. This has the advantage that the energy obtained in the condensation is utilized and not simply released to the environment as in the prior art.

The present invention is hereinbelow elucidated with reference to figures. The figures are for illustration but are not to be understood as limiting.

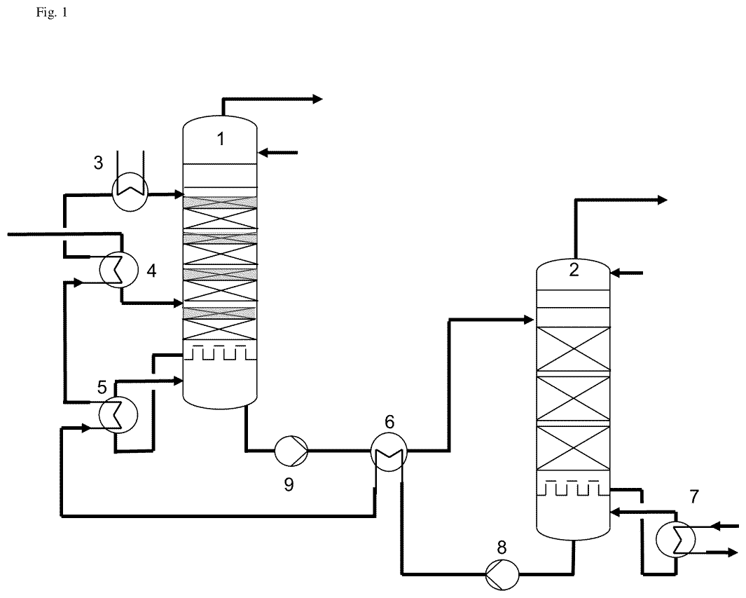

FIG. 1 shows the one embodiment according to the state of the art (WO 2022/161869 A1). The liquid C4-hydrocarbon stream is evaporated via a heat exchanger (4) and passed into the absorber (1). The solvent is-where necessary-brought to the desired temperature via a residual cooler (3) and likewise passed into the absorber, wherein the inlet is spatially above the inlet for the C4-hydrocarbon stream, in the present case above the first random-packing bed. The stream enriched in butanes, which is withdrawn, is obtained at the top of the absorber (1). A possible condensation is not shown here, merely the recycling of a possible substream is indicated. The laden solvent is collected in the bottom of the absorber (1) as indicated by the chimney tray in the figure. There, at least a portion of the laden solvent is withdrawn and passed to the bottom of the absorber (1) via an absorber evaporator (5). The laden solvent is withdrawn from the bottom of the absorber (1) and using a pump (9) passed via the heat exchanger (6) for preheating of the laden solvent to the desorber (2) where the butenes present in the solvent are separated from the solvent. The stream enriched in butenes is obtained at the top of the desorber. This stream may be subjected to a single-or multi-stage condensation which is not shown in the figure. Only a possible recycle stream is indicated. The solvent at least partially freed of butenes is collected in the bottom of the desorber (2) as indicated by the chimney tray in the figure. There, at least a portion of the laden solvent is withdrawn and passed to the bottom of the desorber via a desorber evaporator (7). The solvent freed of butenes is then withdrawn from the bottom of the desorber (2) and using a pump (8) recycled to the absorber via the heat exchanger (6) for preheating the laden solvent, the absorber evaporator (5), the heat exchanger (4) for evaporating the C4-hydrocarbon stream and the residual cooler (3).

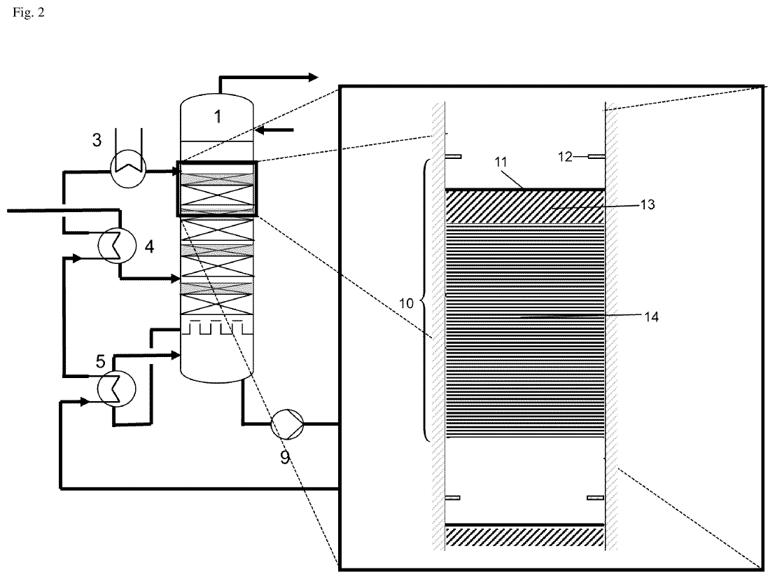

FIG. 2 shows the particularly preferred inventive embodiment where different random packings are present in each random-packing bed in the absorber (1). The further implementation is as described in FIG. 1. The random packings in the upper greyish region of the random-packing bed have a specific surface area y and the random packings in the lower region of the random-packing bed have a specific surface area x, wherein the specific surface area x is greater than the specific surface area y.

FIG. 3 shows a section of FIG. 2 where the construction of the random-packing bed (10) is shown in more detail. In this case the random-packing bed comprises not only a supporting ring (12) for a liquid distributor but also a hold-down grate (11) which is disposed on the random packings (13, 14) and keeps the dumped random packing in the random-packing bed. The upper region of the random-packing bed comprises the random packings (13) of lower specific surface area y. The lower portion of the random-packing bed comprises the random packings (14) of greater specific surface area x.

1. Process for separation of butenes from a C4-hydrocarbon stream which contains at least butenes and butanes by extractive distillation with a solvent, wherein the process comprises the steps of:
a. supplying the gaseous C4-hydrocarbon stream and the liquid solvent to an absorber which comprises at least three random-packing beds arranged one below the other and in which the C4-hydrocarbon stream and the solvent are contacted with one another to transfer predominantly butenes from the C4-hydrocarbon stream into the solvent to form a laden solvent, wherein the thus-laden solvent is collected in a liquid collector of the absorber and passed through an absorber evaporator and then passed into the bottom of the absorber below the liquid collector to outgas predominantly butanes from the laden solvent and wherein the laden solvent is subsequently passed to a desorber as bottoms stream;
b. supplying the laden solvent to the desorber, the bottom of which is at an elevated temperature and preferably a lower pressure relative to the bottom of the absorber and in which the butenes are separated from the solvent to obtain at the top of the desorber a stream enriched in butenes, wherein a solvent, at least partially freed of butenes, is collected in a liquid collector of the desorber and passed through a desorber evaporator and then passed into the bottom of the desorber below the liquid collector to outgas any butenes still remaining in the solvent and wherein the solvent is subsequently recycled to the absorber as bottoms stream;
characterized in that at least one of the random-packing beds of the absorber contains different random packings, wherein the random packings in the upper region of the random-packing bed have a specific surface area y and the random packings in the lower region of the random-packing bed have a specific surface area x,
wherein the specific surface area x is greater than the specific surface area y.

2. Process according to claim 1, wherein the at least one random-packing bed having the different random packings is the uppermost random-packing bed of the absorber.

3. Process according to Claim wherein at least two or three random-packing beds of the absorber contain different random packings.

4. Process according to claim 3, wherein the two or three random-packing beds having different random packings are the uppermost two or the uppermost three random-packing beds of the absorber.

5. Process according to claim 1, wherein all random-packing beds in the absorber contain different random packings.

6. Process according to claim 1, wherein not more than 20% of the volume of the random-packing bed is filled with the random packings of specific surface area y.

7. Process according to claim 1, wherein the employed solvent is NMP.

8. Process according to claim 1, wherein the solvent/the NMP contains water and the water content is between 1% and 10% by weight, preferably between 4% and 9% by weight.

9. Process according to claim 1, wherein the temperature in the bottoms stream of the absorber which is passed to the desorber is between 70° C. and 130° C., preferably 85° C. to 120° C.

10. Process according to claim 1, wherein the temperature in the bottom of the desorber is between 120° C. and 200° C., preferably 130° C. and 195° C.

11. Process according to claim 1, wherein the overhead pressure in the desorber is between 1 and 6 bar absolute, preferably 2 to 5 bar absolute.

12. Process according to claim 1, wherein the preheating of the laden solvent passed to the desorber is performed in two stages, wherein a first heat transfer to the solvent is effected in a heat exchanger and a second heat transfer to the solvent is effected in a kettle evaporator.

13. Process according to claim 1, wherein the heat for evaporation in the desorber evaporator is introduced by heat transfer in a heat exchanger with a suitable heat transfer medium, in particular heating steam.

14. Process according to claim 1, wherein the employed heating steam undergoes at least partial condensation in the heat exchanger, thus generating a hot condensate at a pressure of 10 to 20 bar absolute, preferably 12 to 17 bar absolute, and a temperature of 150° C. to 210° C., preferably 160° C. to 200° C., which is passed to a condensate container.

15. Process according to claim 1, wherein the heating steam for the desorber evaporator is provided using a steam ejector supplied with high pressure or medium pressure steam and the low pressure steam obtained in the condensate container.
